(19) United States
(12) Patent Application Publication
(10) Pub. No.: US 2019/0183832 A1
(43) Pub. Date: Jun. 20, 2019

(54) NUTRITIONAL BOLUS FOR ANIMALS

(71) Applicant: Michael Guarnieri, Baltimore, MD (US)

(72) Inventor: Michael Guarnieri, Baltimore, MD (US)

(21) Appl. No.: 16/219,554

(22) Filed: Dec. 13, 2018

Related U.S. Application Data
(60) Provisional application No. 62/608,303, filed on Dec. 20, 2017.

(51) Int. Cl. A61K 31/215; A61P 3/04; A61P 41/00; A23L 33/12; A61K 31/20

(52) U.S. Cl. CPC A61K 31/215; A61P 3/04; A61K 31/20; A23L 33/12; A61P 41/00

(57) ABSTRACT

Formulations and methods for reducing or preventing stress-related weight loss in animals have been developed. The formulations include medium-chain triglyceride (MCT) oil as the active agent. The methods include subcutaneously administering the formulation to an animal in need thereof shortly before, during, or after the stress. The stress may be due to restraint, surgery, injury, transportation, relocation, birth, separation, exercise, training, handling, rescue or other action that elicits stress or high energy expenditure or involves an animal with low energy reserves, such as a newborn or sick animal. The animals are typically agriculture animals, laboratory animals, veterinary animals, zoo animals, or pets. Also provided are kits containing ready-to-use dosage forms for reducing or preventing stress-related weight loss in animals.

### CROSS-REFERENCED TO RELATED APPLICATIONS

This application claims the benefit of and priority to U.S. Provisional Application No. 62/608,303 filed Dec. 20, 2017, which is hereby incorporated herein by reference its entirety.

### FIELD OF THE INVENTION

The invention is generally directed to animal food supplements and to methods for providing rapid nutritional intervention to animals.

### BACKGROUND OF THE INVENTION

Millions of animals are injured yearly or acquire a disease needing veterinary care and nutritional relief. Injuries such as facial injury, gastro intestinal obstruction, drug-inhibition of intestinal motility, loss of appetite secondary to a disease, or recovery from surgery can all cause decreased ingestion of food. Weight loss can inhibit recovery from broken bones, internal injuries, and surgery. Newborns often are too weak to nurse or their mothers do not have sufficient milk at birth, causing weakness and even death without immediate intervention.

Nutrition must be provided to animals who cannot feed themselves. Veterinarians can introduce a gastric tube to force nutrients into the stomach. Alternatively, food and water can be supplied to sedated or restrained animals through intravenous (IV) or subcutaneous (SC) infusions.

Current nutritional approaches are time consuming and require high levels of veterinary experience. Gastric tubes cannot be used for animals with facial injuries. The IV or SC methods are the most common alternative. The caretaker restrains the animal, exposes a vein, and slowly infuses fluids into the vein or places a needle under the skin to infuse fluids.

Infusions overall are time consuming, personnel intensive, and have high rates of adverse events including infection and edema secondary to inflammatory reactions in the vein and SC space. The insertion of a gastric tube requires significant experience. Iatrogenic injury rates caused by insertion and removal of the tube are high. Emergency feeding methods using gastric tubes and IV or SC infusions are also slow. Even with restraints in place and sedation, IV and SC feeding techniques can injure the animal, damage veins, and cause infections. The process of restraining and sedating the animal can lead to significant bite and scratch wounds among care providers.

There remains a need for rapid nutritional intervention delivering high calorie nutrition to animals under stress.

Therefore, it is the object of the present invention to provide a formulation for rapid nutritional intervention in animals under stress.

It is another object of the present invention to provide methods for rapid nutritional intervention in animals under stress.

It is yet another object of the present invention to provide dosage forms for rapid nutritional intervention in animals under stress.

### SUMMARY OF THE INVENTION

Formulations and methods for rapid nutritional intervention in distressed animals are provided. The formulations include oils with saturated, mono- and poly-unsaturated fatty acids that are liquid at room temperature. Typically, the formulations include a sterile medium-chain triglyceride (MCT) oil as the active agent. The MCT oils include triglycerides with saturated and unsaturated fatty acids having fatty acid chain length between four (C₄) and 18 (C₁₈) carbons. The MCT oils may also include triglycerides with mono- and poly-unsaturated long chain fatty acids having a fatty acid chain length between C₁₄ and C₂₄ and which are liquid at room temperature. The MCT oils typically include at least one fatty acid with a fatty acid chain length between C₆ and C₁₂ carbons.

The formulations may contain a second agent. The second agent may be a nutraceutical or a pharmaceutical agent. Suitable nutraceutical or a pharmaceutical agents include vitamins, minerals, antibiotics, anti-parasiticals, and analgesics.

The MCT oil in the formulations is not a vehicle for delivering the second agent, or for improving bioavailability or biodistribution of the second agent. The formulations may be substantially free of analgesics, antibiotics, and anti-parasitical agents. The formulations may be substantially free of cholesterol. The formulations may be substantially free of glycerol. The formulations may be substantially free of glycerol tristearate. The formulations may be substantially free of cholesterol, glycerol, and glycerol tristearate.

The methods include subcutaneously injecting a bolus of the formulation into distressed animals. Typically, the SC injections are fast, as compared to the time needed for IV or SC infusions delivering nutrition with about the same calorie content. The methods require minimal labor skills and may be practiced by any caregiver, veterinary, zoo or farm staff, or a pet owner. The methods also minimize the adverse events associated with handling, restraining, or injection procedures during IV or SC infusions.

The methods reduce or prevent stress-related weight loss in small to medium size animals. Small to medium size animals include animals weighing between 0.01 kg (0.02 lb) and 30 kg (66 lb). Examples of animals that may benefit from the methods include animals in agriculture, zoo animals, laboratory animals, domestic pets, animals in shelters, and animals cared for at veterinary clinics.

The stress-related weight loss may be due to a drop in appetite, inability to feed, or incapacitation. The stress may be caused by restraint, illness, surgery, injury, transportation, relocation, birth, separation, exercise, training, handling, and rescue.

The formulations and methods typically deliver between about 0.1 ml/kg and about 15 ml/kg dose of MCT oil to an animal. The dose may vary based on the size of the animal and the amount of calories needed for rapid nutritional intervention. The volume of the formulations and rate of the SC injections may be limited by the size of the animal, and the SC injections may be repeated.

The formulations are not food supplements for long-term dietary needs. The formulations are not designed to supplement a non-distressed animal's food intake, nor for long-term use to improve animal's mental health or cognition. The formulations and methods are for a specific use: for rapidly providing nutrition to an animal that has been, is, or will be subject to stress.

Kits and ready-to-use dosage forms, such as sterile syringes pre-loaded with the formulations, are provided. These may include septum capped vials holding between 5 and 500 ml of the formulations.

### DETAILED DESCRIPTION OF THE INVENTION

### I. Definitions

As used herein, the term “stress” refers to a physical and/or emotional state of an animal induced by an adverse action to the animal. Examples of adverse actions inducing stress include restraint, surgery, illness, injury, transportation, relocation, birth, separation, exercise, training, handling, and the process of rescue.

As used herein, the term “stress-related weight loss” refers to the loss in an animal's body weight induced by stress. Examples of stress-related weight loss include weight loss due to inability to feed, loss of appetite to feed, fear, dehydration, overheating, disorientation, and hostility. Stress-related weight loss may occur even when the animal has free access to food and water.

As used herein, the term “animal in need” or “subject in need” as used herein refers to a small to medium size animal, such as an animal between 0.01 kg and 30 kg in weight. Examples of animals or subjects include a non-human animal such as a primate, non-human primate, laboratory animal, farm animal, livestock, or a domestic pet.

As used herein, the terms “effective amount” or “therapeutically effective amount” refers to a dosage sufficient to reduce or prevent weight loss. The precise dosage will vary according to a variety of factors such as subject-dependent variables (e.g., age, gender, weight, etc.) and the severity of the stress.

The term “reduce”, in the context of weight loss, refers to reducing or decreasing weight loss. Reduction can be compared to a control animal's weight or to a standard level. Comparing to a control animal's weight is comparing to the weight of an animal of the same species, strain, gender, and age. The control animal may be a stress-free animal not experiencing weight loss, and the weight of this animal is the “stress-free control weight”. The control animal may be an untreated animal that experienced stress-related weight loss and lost between 1% and 25% of its pre-stress weight. The weight of this animal is the “stress-related control weight”.

As used herein, the term “prevention” or “preventing”, in the context of weight loss, refers to stopping weight loss from occurring. Prevention of weight loss is detected when the weight of the animal before and some short time after the adverse action are substantially similar. The animal's weight during a short time after stress is substantially similar to the stress-free control weight. A short time may be 1 hour, 2 hours, 3 hours, 6 hours, 12 hours, 24 hours, two days, three days, four days, five days, six days, or one week. In some aspects, the short time may be about 1 hour, 2 hours, 3 hours, 6 hours, 12 hours, or 24 hours.

As used herein, the term “active agent”, refers to a therapeutically or pharmacologically active substance that acts locally or systemically in the body to reduce or prevent weight loss.

As used herein, the term “second agent” refers to a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

As used herein, the term “vehicle” refers to a carrier or inert substance used as a solvent (or diluent) in which an active agent is formulated, diluted, and/or administered.

The terms “sufficient” and “effective”, as used interchangeably herein, refer to an amount (e.g. mass, volume, dosage, concentration, and/or time period) needed to achieve one or more desired result(s), such as reduction or prevention of weight loss.

As used herein, the term “substantially similar” or “substantially unchanged” refers to two or more values, physiological states, physical states, or health conditions that are about the same, or not significantly different from one another.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%.

### II. Formulations

The formulations include MCT oils that are liquid at room temperature. The MCT oil is the active agent of the formulations. The formulations may include a second agent.

A. MCT Oil Formulations

The formulations include oils with similar properties to medium chain triglycerides (MCT) having fatty acids with fatty acid chain lengths between C₆ and C₁₂. The oils may include fatty acids with fatty acid chain length between four (C₄) and 24 (C₂₄) carbons as the active agent. The composition of MCT oils may vary. The fatty acids may be saturated fatty acids. The fatty acids may be mono-unsaturated fatty acids. The fatty acids may be poly-unsaturated fatty acids. The formulations are liquids at room temperature (about 20° C., about 23° C., or between about 20° C. and 25° C.).

1. MCT Oil

Oils with similar properties to medium chain triglycerides can be used including C4 to C10 saturated fatty acids; C6-C24 mono and poly unsaturated acids that are liquid at room temperature. MCT oils may have different proportions of fatty acid with fatty acid chain lengths between C4 and C24.

Generally, the MCT oil may contain any one of the following fatty acids: C4:0 (Butanoic acid), C6:0 (caproic acid), C8:0 (caprylic acid; octanoic acid), C10:0 (capric acid; decanoic acid), C12:0 (lauric acid), C14:0 (myristic acid), C16:0 (palmitic acid), C16:1 (Palmitoleic acid), C18:0 (stearic acid), C18:1 (Elaidic acid, trans, in hydrogenated vegetable), C18:1 (Oleic acid, cis), C18:1 (Vaccenic acid, trans, in dairy products), C18:2 (Linoleic acid, cis), C18:2 (Linolelaidic acid, trans, in partially hydrogenated vegetable oil), C18:3 (α-Linolenic acid, cis, in flaxseed), C18:3 (γ-Linolenic acid, cis, in black currant oil), C18:4 (Stearidonic acid), C20:1 (Gondoic acid, cis, in jojoba oil), C20:1 (Paullinic acid, cis, in guarana), C20:3 (Dihomo-γ-linolenic acid), C20:3 (Mead acid), C20:4 (Arachidonic acid), C20:5 (Eicosapentaenoic acid), C22:1 (Erucic acid), C22:4 (Docosatetraenoic acid), C22:6 (Docosahexaenoic acid), and C24:1 (Nervonic acid).

Any one of these fatty acids may constitute between 1% and 99% of the total fat of the oil, such as between 10% and 80%, 20% and 70%, 30% and 70%, 40% and 70% of the total fat of the oil. Exemplary oils may include any one of the fatty acids at about 80%, about 70%, about 60%, about 50%, about 40%, about 30%, about 20%, about 10%, about 5%, or about 1% of the total fat of the oil.

For example, a MCT oil may include the following fatty acids as percent of total fat: about 1% of C6:0 (caproic acid), about 75% of C8:0 (caprylic acid; octanoic acid), about 23% of C10:0 (capric acid; decanoic acid), about 1% of C12:0 (lauric acid), and a trace of C18:0 (stearic acid). Other MCT oils may include these same triglycerides in different proportions. Other MCT oils may include fatty acids such as a trace of C14:0 (myristic acid), C16:0 (palmitic acid), C18:1 (oleic acid), and C18:2 (linoleic acid).

MCT oils may be formed to have a larger percentage of lauric fatty acid content. For example, an MCT oil may have about 32% C12:0 (lauric), about 24% C8:0 (caprylic), about 28.5% C10:0 (capric), about 7% C18:1 (oleic), and traces of other unsaturated fatty acids.

Other examples of MCT oil include MCT oil containing about 2.0% C6:0 (Caproic acid); about 65.0-80.0% C8:0 (Caprylic acid); about 20.0-35.0% C10:0 (Capric acid); about 2% C12:0 (Lauric acid); and about 1.0% C14:0 (Myristic acid).

Other examples of MCT oil include MCT oil containing about 2.0% C6:0 (Caproic acid); about 50.0-65.0% C8:0 (Caprylic acid); about 30.0-45.0% C10:0 (Capric acid); about 2% C12:0 (Lauric acid); and about 1.0% C14:0 (Myristic acid).

Other components in the MCT oil include trace amounts of water, metals, such as heavy metals, and ash.

Generally, the formulations contain at least 50% by weight medium-chain triglyceride oil. In some aspects, the formulations consist only of medium-chain triglyceride oil. In some aspects, the formulations contain medium-chain triglyceride oil and are substantially free of, or do not contain, a second agent.

MCT oil is commercially available in grades that meet US Food and Drug Administration (FDA) standards for cosmetics, foods, and drugs. The manufacture was first reported about 1958 by Drew Chemical Corporation. Chemists liberated free fatty acids by steam hydrolysis of coconut oil and distilled the fraction containing 6, 8, and 10-carbon long fatty acids: C6 (caproic acid), C₈ (caprylic acid; octanoic acid) and C₁₀ (capric acid; decanoic acid).

a. Caloric Content

The oils from the injected formulation are transported to the liver where they are degraded to 2-carbon fragments and completely oxidized to rapidly supply nutrition to the distressed animal. Typically, the volume of the injected formulation varies with the size of the animal and the amount of calories needed for rapid nutritional intervention.

The term “calories” refers to food calories, which are kilocalories, kcal, used in food industry simply as calories, or “cal”. Typically, a 10 ml of formulation provides between about 70 and 150 calories, such as about 80 calories, about 90 calories, about 100 calories, about 110 calories, or about 120 calories to an animal.

For example, 10 ml of a subcutaneously injected formulation may provide about 70 calories, 75 calories, 80 calories, 85 calories, 90 calories, 95 calories, 100 calories, 105 calories, 110 calories, 115 calories, 120 calories, 125 calories, 130 calories, 135 calories, 140 calories, 145 calories, or about 150 calories to an animal.

In an exemplary method, an injection of 5 mL of the formulation provides approximately 50 calories of nutritional energy. For reference, this therapy in a typical cat is comparable to infusing the average human adult with the caloric equivalent of a McDonalds Big Mac. A SC injection of a 5 mL of the formulation in a typical cat would take about 1 min to complete, as opposed to IV infusions, which take hours to complete to deliver the same amount of calories.

b. Density

Generally, the density of the formulations is similar to MCT oil density. The formulations may have a density at 20° C. between about 0.93 g/cm³ (g/ml) and 0.96 g/cm³ (g/ml). The formulation may have a specific density at 20° C. of about 0.93 g/cm³, about 0.94 g/cm³, about 0.95 g/cm³, or about 0.96 g/cm³.

c. Viscosity

Generally, the viscosity of the formulations at 20° C. is between about 25 mPa·s (cP) and 130 mPa·s (cP). The formulation may have a specific viscosity at 20° C. of about 25 cP, about 30 cP, about 35 cP, about 40 cP, about 45 cP, about 50 cP, about 55 cP, about 60 cP, about 65 cP, about 70 cP, about 75 cP, about 80 cP, about 85 cP, about 90 cP, about 95 cP, about 100 cP, about 105 cP, about 110 cP, about 115 cP, about 120 cP, about 125 cP, or about 130 cP. Preferred viscosities for the formulations include between about 20 cP and about 100 cP, such as between about 40 cP and 60 cP.

d. Resuspendability and Syringeability

The term resuspendability refers to the ability of a composition to form a uniform suspension. In during manufacturing, solids are uniformly blended with liquids to generate a uniform suspension. If the solids settle out of the suspension during storage, the manufacturer claims that uniformity can be reestablished by gentle to vigorous agitation. If the uniform suspension cannot be established by the resuspension instructions, the product does not conform. The formulations are typically resuspended to a uniform suspension without agitation. The formulations may be resuspended to a uniform suspension by gentle to vigorous agitation, such as shaking, by hand or machine, and for seconds to minutes of more.

The term syringeability refers to the ability of a liquid, gel, or semi-solid product to be injected via a syringe and needle. The term is frequently combined with the volume of the syringe and gauge (G) of the needle. For example, some oils can be delivered easily through a large gauge needle (14-20 G), through lower gauge needles, such as 20-22 G, but practically cannot be delivered through a smaller 26-28 G needle. Other oils may be delivered through smaller needles, such as between 22G and 28G. The formulations may be easily delivered with syringes between 10 and 31 G, such as with syringes between 14G and 28G.

2. Second Agent

The formulations may contain a second agent. Suitable second agents include vitamins, minerals, antibiotics, anti-parasiticals, and analgesics.

In some aspects, the formulations may include at least 50% by weight (wt %) medium-chain triglyceride oil and at least 0.1% wt % of the second agent.

The formulations may include between 0.1 wt % and 50 wt % of the second agent. The second agent may be present at about 0.1 wt %, about 0.3 wt %, about 0.5 wt %, about 0.8 wt %, about 1 wt %, about 2 wt %, about 3 wt %, about 4 wt %, about 5 wt %, about 6 wt %, about 7 wt %, about 8 wt %, about 9 wt %, about 10 wt %, about 15 wt %, 20 wt %, 25 wt %, about 30 wt %, about 35 wt %, about 40 wt %, about 45 wt %, or about 50 wt % of the total formulation. For example, the formulation may contain between about 0.1 wt % and 5 wt % of the second agent. Preferably, the second agent is present at a concentration between about 0.1 wt % and 3 wt %, such as between about 1 wt % and 2 wt %.

The MCT oil in the formulations is not a vehicle for delivering the second agent, or for improving bioavailability or biodistribution of the second agent. The formulations may be substantially free of second agents, such as analgesics, antibiotics, and anti-parasitical agents. The formulations may be substantially free of cholesterol. The formulations may be substantially free of glycerol. The formulations may be substantially free of glycerol tristearate. The formulations may be substantially free of cholesterol, glycerol, and glycerol tristearate.

a. Vitamins and Minerals

Examples of oil-soluble vitamins include vitamins A, D, and E. Vitamin A group includes, retinol, 3-dehydroretinol, retinal, 3-dehydroretinal, retinoic acid, 3-dehydroretinoic acid, and esters such as vitamin A acetate and vitamin A palmitate and, as provitamin A, carotenoids and xanthophylls such as α-, β-, and γ-carotenes, β-cryptoxanthin, and echinenone. Examples of vitamin D include vitamin D2 to D7. Examples of vitamin E include α-, β-, γ-, and δ-tocopherols, and α-, β-, γ-, and δ-tocotrienols and esters such as vitamin E acetate and vitamin E nicotinate. Examples of vitamin K include vitamin K1 to K3.

Examples of minerals include aluminum and iron containing minerals.

In some aspects, the formulations may include at least 50% by weight (wt %) medium-chain triglyceride oil and at least 0.1% wt % of a vitamin or a mineral. The formulations may include between 0.1 wt % and 50 wt % of the vitamin or a mineral. The vitamin or a mineral may be present at about 0.1 wt %, about 0.3 wt %, about 0.5 wt %, about 0.8 wt %, about 1 wt %, about 2 wt %, about 3 wt %, about 4 wt %, about 5 wt %, about 6 wt %, about 7 wt %, about 8 wt %, about 9 wt %, about 10 wt %, about 15 wt %, 20 wt %, 25 wt %, about 30 wt %, about 35 wt %, about 40 wt %, about 45 wt %, or about 50 wt % of the total formulation. For example, the formulation may contain between about 0.1 wt % and 5 wt % of the vitamin or mineral. Preferably, the vitamin or mineral is present at a concentration between about 0.1 wt % and 3 wt %, such as between about 1 wt % and 2 wt %.

b. Anti-Microbial and Anti-inflammatory Agents

Examples of anti-microbial agents include agents against viral, bacterial, or fungal infection, such as, neomycin, mitomycin, plicamycin, bleomycin, streptomycin, chloramphenicol, cephalosporin, ampicillin, penicillin, tetracycline, and ciprofloxacin griseofulvin, ketoconazole, itraconizole, amphotericin B, nystatin, and candicidin, antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, and methdilazine); antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., interferon alpha, beta or gamma, zidovudine, amantadine hydrochloride, ribavirin, and acyclovir); antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin); corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, hydrocortisone sodium succinate; or a combination of two or more of these agents.

Specific examples of antibiotics and antiinflammatories include glucocorticosteroids such as dexamethasone, cortisone, prednisone, hydrocortisone, beclomethasone dipropionate, betamethasone, flunisolide, methylprednisone, paramethasone, prednisolone, triamcinolone, alclometasone, amcinonide, clobetasol, fludrocortisone, diflorasone diacetate, fluocinolone acetonide, fluocinonide, fluorometholone, flurandrenolide, halcinonide, medrysone and mometasone and pharmaceutically acceptable mixtures and salts thereof, and non-steroidal antiinflammatories such as rimadyl, and aspirin.

In some aspects, the formulations may include at least 50% by weight (wt %) medium-chain triglyceride oil and at least 0.1% wt % of an antimicrobial or antiinflmmatory agent. The formulations may include between 0.1 wt % and 50 wt % of the antimicrobial or anti-inflammatory agent. The antimicrobial or anti-inflammatory agent may be present at about 0.1 wt %, about 0.3 wt %, about 0.5 wt %, about 0.8 wt %, about 1 wt %, about 2 wt %, about 3 wt %, about 4 wt %, about 5 wt %, about 6 wt %, about 7 wt %, about 8 wt %, about 9 wt %, about 10 wt %, about 15 wt %, 20 wt %, 25 wt %, about 30 wt %, about 35 wt %, about 40 wt %, about 45 wt %, or about 50 wt % of the total formulation. For example, the formulation may contain between about 0.1 wt % and 5 wt % of the antimicrobial agent. Preferably, the antimicrobial agent is present at a concentration between about 0.1 wt % and 3 wt %, such as between about 1 wt % and 2 wt %.

c. Anti-Parasitic Agents

Examples of anti-parasitic agents include nitazoxanide, melarsoprol, eflornithine, metronidazole, tinidazole, miltefosine, mebendazole, pyrantel pamoate, thiabendazole, diethylcarbamazine, ivermectin, niclosamide, praziquantel, albendazole, praziquantel, rifampin, amphotericin B, imidacloprid, moxidectin, fipronil, methoprene, eprinomectin, praziquantel, selamectin, sarolaner, afoxolaner, milbemycin oxime, milbemycin oxime, spinosad, lufenuron, or a combination of two or more of these agents.

In some aspects, the formulations may include at least 50% by weight (wt %) medium-chain triglyceride oil and at least 0.1% wt % of an anti-parasitic agent. The formulations may include between 0.1 wt % and 50 wt % of the anti-parasitic agent. The anti-parasitic agent may be present at about 0.1 wt %, about 0.3 wt %, about 0.5 wt %, about 0.8 wt %, about 1 wt %, about 2 wt %, about 3 wt %, about 4 wt %, about 5 wt %, about 6 wt %, about 7 wt %, about 8 wt %, about 9 wt %, about 10 wt %, about 15 wt %, 20 wt %, 25 wt %, about 30 wt %, about 35 wt %, about 40 wt %, about 45 wt %, or about 50 wt % of the total formulation. For example, the formulation may contain between about 0.1 wt % and 5 wt % of the anti-parasitic agent. Preferably, the anti-parasitic agent is present at a concentration between about 0.1 wt % and 3 wt %, such as between about 1 wt % and 2 wt %.

d. Analgesic Agents

Examples of analgesics include any of the opioids such as buprenorphine and butorphanol, or drugs such as fentanyl. Representative local anesthestics include bupivacaine, ropivacaine, dibucaine, procaine, chloroprocaine, prilocaine, mepivacaine, etidocaine, tetracaine, lidocaine, and xylocaine, and mixtures thereof can also be used, alone or in combination with other analgesics. Preferred analgesics include buprenorphine and butorphanol, or drugs such as fentanyl.

In some aspects, the formulations may include at least 50% by weight (wt %) medium-chain triglyceride oil and at least 0.1% wt % of an analgesic agent. The formulations may include between 0.1 wt % and 50 wt % of the analgesic agent. The analgesic agent may be present at about 0.1 wt %, about 0.3 wt %, about 0.5 wt %, about 0.8 wt %, about 1 wt %, about 2 wt %, about 3 wt %, about 4 wt %, about 5 wt %, about 6 wt %, about 7 wt %, about 8 wt %, about 9 wt %, about 10 wt %, about 15 wt %, 20 wt %, 25 wt %, about 30 wt %, about 35 wt %, about 40 wt %, about 45 wt %, or about 50 wt % of the total formulation. For example, the formulation may contain between about 0.1 wt % and 5 wt % of the analgesic agent. Preferably, the analgesic agent is present at a concentration between about 0.1 wt % and 3 wt %, such as between about 1 wt % and 2 wt %.

e. Nutrients

The formulations may include essential nutrients including lipids and protein components. Essential lipids not otherwise listed include poly unsaturated fatty acids. Essential protein components include natural and synthetic d/1 amino acids.

f. Preservatives, Antioxidants, other Excipients

The formulations may include a preservative, antioxidant or other inactive excipient such as a pH buffer.

A preservative inhibits the deterioration and/or decomposition of the formulation. Deterioration or decomposition can be brought about by any of microbial growth, fungal growth, and undesirable chemical or physical changes. Suitable preservatives include benzoate salts (e.g. sodium benzoate), ascorbic acid, methyl hydroxybenzoate, ethyl p-hydroxybenzoate, n-propyl p-hydroxybenzoate, n-butyl p-hydroxybenzoate, potassium sorbate, sorbic acid, proprionate salts (e.g. sodium propionate), chlorobutanol, benzyl alcohol, and combinations thereof. Typically, the preservative constitutes less than 2% wt/wt, between about 0.01% wt/wt and about 1.5% wt/wt, inclusive, between about 0.1% wt/wt and about 1% wt/wt, inclusive, between about 0.1% wt/wt and about 0.5% wt/wt, inclusive, or about 0.3% wt/wt of the formulation. Preferred preservatives include benzyl alcohol at about 1% wt/wt.

B. Formulations

1. Sterilization

Any of the formulations may be sterilized to minimize infections and provide long term storage of the formulation.

The sterile formulations may be formed with the inclusion of one or more preservatives in the formulation. The sterile formulations may also be formed after ionizing radiation, such as gamma radiation sterilization, or filter sterilization.

2. Storage and Stability

Typically, the formulations are stable at room temperature and at elevated temperatures for at least about a month, six months, a year, or two years.

The stability of the formulations may be determined by comparing any one or more of the viscosity, density, resuspendability, sterility, and syringeability of the formulations at 20° C. at the time of preparation and after a period of storage at 20° C., 25° C., 30° C., 40° C., or 50° C. The storage period may be one week, two weeks, three weeks, one month, two months, three months, four months, five months, six months, seven months, eight months, nine months, ten months, 11 months, 12 months, one year, or two years.

The formulation may be considered stable if following storage the value for viscosity, density, resuspendability, sterility, and/or syringeability of the formulation has changed by less than 10% over the value for viscosity and/or density of the formulation at the time of preparation.

Typically, the formulations are stable at 20° C., 25° C., 30° C., 40° C., or 50° C. for at least about 1 month, for at least a period of time between one month and six months, or for at least a period of time between one month and 24 months. The sterile formulations may remain stable when stored in septum-capped drug vials with 1% benzyl alcohol preservative at any of 20° C., 25° C., or 40° C. for at least a period of time between one month, for at least a period of time between one month and six months, for at least a period of time between one month and 24 months.

3. Dosages and Dosage Forms

The formulations may be presented in one or more dosage forms. The dosage forms may be selected by the end user to best meet the needs of a particular animal.

The formulations and methods typically deliver between about 0.1 ml/kg and about 15 ml/kg dose of MCT oil to an animal. The dose may vary based on the size of the animal and the amount of calories needed for rapid nutritional intervention. For example, a 10 ml volume of the formulation delivers about 100 calories to an animal. Dependent on the nutritional intervention needed, the dose of about 1.5 ml/kg may be used to deliver 10 ml of the formulation to a cat, providing about 100 calories of nutritional energy.

Examples of dosages for animals of different weights are provided in Table 1.

TABLE 1
Examples of animal weights and associated
dosages suitable for SC injections.

| | | | Volume | | | | |
|---|---|---|---|---|---|---|---|
| | | | MCT oil | | | | |
| | Weight | Weight | injected | Dosage | | Dosage | |
| | (kg) | (lb) | (ml) | ml/kg | ml/lb | mg/kg | mg/lb |
| Mouse | 0.02 | 0.04 | 0.25 | 12.50 | 6.25 | 12.00 | 6.00 |
| Rat | 0.12 | 0.26 | 1 | 8.33 | 3.85 | 8.00 | 3.69 |
| Cat | 6.8 | 15 | 10 | 1.47 | 0.67 | 1.41 | 0.64 |
| Dog | 22.7 | 50 | 10 | 0.44 | 0.20 | 0.42 | 0.19 |

Suitable volumes for SC injections of the formulations include about 0.1 ml, 0.2 ml, 0.3 ml, 0.4 ml, 0.5 ml, 0.6 ml, 0.7 ml, 0.8 ml, 0.9 ml, 1 ml, 1.2 ml, 1.3 ml, 1.5 ml, 1.6 ml, 1.7 ml, 1.8 ml, 1.9 ml, 2 ml, 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, 10 ml, 10.5 ml, 11 ml, 11.5 ml, 12 ml, 12.5 ml, 13 ml, 13.5 ml, 14 ml, 14.5 ml, or 15 ml. Preferred volumes include between about 0.2 ml and 10 ml of the formulations, such as between 0.2 ml and 8 ml, 0.2 ml and 5 ml, 0.2 ml and 3 ml, 0.2 ml and 2 ml, or about 1 ml of the formulation.

Suitable dosages for SC injections include about 0.1 mg/kg, about 0.3 mg/kg, about 0.5 mg/kg, about 0.8 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or about 15 mg/kg. Preferred dosages include between 0.2 mg/kg and 15 mg/kg, such as between 0.2 mg/kg and 12 mg/kg, 0.2 mg/kg and 10 mg/kg of the formulations, such as between about 0.2 mg/kg and 8 mg/kg, 0.2 mg/kg and 5 mg/kg, 0.2 mg/kg and 3 mg/kg, 0.2 mg/kg and 2 mg/kg, or about 1 mg/kg of the formulation.

Suitable dosages for SC injections include about 0.1 ml/kg, about 0.3 ml/kg, about 0.5 ml/kg, about 0.8 ml/kg, about 1 ml/kg, about 2 ml/kg, about 3 ml/kg, about 4 ml/kg, about 5 ml/kg, about 6 ml/kg, about 7 ml/kg, about 8 ml/kg, about 9 ml/kg, about 10 ml/kg, or about 15 ml/kg. Preferred dosages include between 0.2 ml/kg and 15 ml/kg, such as between 0.2 ml/kg and 12 ml/kg, 0.2 ml/kg and 10 ml/kg of the formulations, such as between about 0.2 ml/kg and 8 ml/kg, 0.2 ml/kg and 5 ml/kg, 0.2 ml/kg and 3 ml/kg, 0.2 ml/kg and 2 ml/kg, or about 1 ml/kg of the formulation.

The dosage forms may be sterile vials, such as sterile storage vials, sterile septum capped vials, containing between 1 ml and 500 ml of formulations. The dosage forms may be sterile single-dose/single-use vials, or sterile multi-dose vials. The sterile single-dose/single-use vials may contain about 1 ml, 1.2 ml, 1.3 ml, 1.5 ml, 1.6 ml, 1.7 ml, 1.8 ml, 1.9 ml, 2 ml, 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, 10 ml, 10.5 ml, 11 ml, 11.5 ml, 12 ml, 12.5 ml, 13 ml, 13.5 ml, 14 ml, 14.5 ml, or 15 ml of the formulation. The sterile multi-dose vials may contain any volume of the formulation from between 5 ml and 500 ml. Preferably, the septum capped vials contain between 5 ml and 50 ml of formulations.

The dosage forms may be ready-to-use pre-loaded syringes. The ready-to-use pre-loaded syringes include sterile capped syringes with a volume between 5 ml and 100 ml pre-loaded with sterile formulations. The ready-to-use dosage forms may be sterile syringes with a volume about 0.3 milliliters (ml), 0.5 ml, 1 ml, 2 ml, 2.5 ml, 3 ml, 5 ml, 6 ml, 10 ml, 12 ml, 20 ml, 50 ml, 60 ml, 70 ml, or 100 ml. The ready-to-use dosage forms may be sterile syringes with a volume between 0.3 ml and 5 ml, pre-fitted with needles, and pre-loaded with sterile formulations.

### III. Methods of Use

A. Methods of Administration

The methods include subcutaneously injecting formulations containing MCT oil as the active agent into small to medium size animals to reduce or prevent stress-related weight loss. The formulations and methods are not intended or formulated to supplement a non-distressed animal's food intake, and are not intended or formulated for long-term use to improve animal's mental health or cognition. The formulations and methods are for a specific use: for rapidly providing nutrition to an animal that has been, is, or will be subject to stress.

The methods resolve at least four problems routinely encountered in the art. First, a bolus SC injection of MCT oil can be complete in seconds compared to the several hours required for IV and SC infusions to deliver the equivalent amount of calories. Second, the need for sedation or restraint—both of which can cause stress and further injury is eliminated. The MCT oil is injected SC into the fur behind the neck, i.e., dorsal mid scapular scruff. This is the fur area that mothers use to collect their pups. The quick injection involves minimal stress for the animal and caretaker. Third, the method requires minimal training. Fourth, SC injections of MCT rescue the nutritional effects of appetite suppression caused by stress, such as by the humane use of post-surgical opiate analgesia. Fifth, extensive histopathology studies demonstrated no adverse effects on the tissues affected by the three SC injections repeated at 4-day intervals of 1 mL of MCT per injection.

In an exemplary method, the caretaker uses sterile syringes with a volume between 5 and 50 mL, sterile disposable needles with a size between 10 and 25 gauge, and a sterile formulation in a septum capped vial. The caretaker uses sterile techniques to fit the needle to the syringe and fill the syringe with the desired amount pharmaceutical grade formulation from the septum capped vial. The injured animal is gently held in a scruff hold and injected in the scruff with the formulation.

Other exemplary methods include using sterile capped syringes with a volume between 5 and 100 mL pre-loaded with a sterile formulation and sterile disposable needles with a size between 10 and 25 gauge. The caretaker selects a syringe with the desired volume of the formulation. The injured animal is gently held in a scruff hold and injected in the scruff with the formulation.

Other exemplary methods include using sterile syringes with a volume between 0.3 and 5 mL, pre-fitted with sterile needles, and pre-loaded with a sterile formulation. The needles may have a size between 25 and 31 gauge. The caretaker selects a syringe with the desired volume of the formulation. The injured animal is gently held in a scruff hold and injected in the scruff with the formulation.

The timing of the SC injection is typically just prior to, during, or shortly after stress. Typically, the SC injection has a short duration and is complete within a period of time between a few seconds and a few minutes. Generally, the formulation is administered to the dorsal mid scapular scruff. Other suitable sites for subcutaneous injection include over the ribs well behind the shoulder, inside the thigh beneath the fold of the skin, beneath the skin behind the shoulder, under the wings (axilla), upper thigh/lateral flank (between the upper part of the leg and the lateral body wall), patagium (wing web), and the space between scapulae. The subcutaneous injection site may differ in different animals. For example, the site may be the shoulder blade area for lizards, the loose skin in the shoulder areas for chelonians, and midway between the muscle groups along either side of the spine on the back and the lateral midline of the body for snakes, under the wings for birds including chickens, and dorsal mid scapular scruff for cats and dogs.

The SC injection is typically administered a short time before, during, and/or a short time after stress. The short time may be 1 hour, 2 hours, 3 hours, 6 hours, 12 hours, 24 hours, two days, three days, four days, five days, six days, or one week. In some aspects, the short time may be about 1 hour, 2 hours, 3 hours, 6 hours, 12 hours, or 24 hours.

The SC injection may be repeated as many times as is necessary. The SC injections may be repeated one, two, three, four, five, six, seven, eight, nine, or more times. The SC injection may be repeated until the animal begins to feed independently.

The duration of administration may vary between different animals, or different calorie requirements for the same animal. The duration of administration may be as short as few seconds or few minutes. The duration of administration may be about 2 seconds (sec), 3 sec, 5, sec, 10 sec, 15 sec, 20 sec, 25 sec, 30 sec, 35 sec, 40 sec, 45 sec, 50 sec, 55 sec, 60 sec, 70 sec, 80 sec, 90 sec, 100 sec, 110 sec, 120 sec, 130 sec, 140 sec, 150 sec, 160 sec, 170 sec, or 180 sec.

For example, a typical cat recovering post-surgery may require 10 ml of MCT oil for 100-calorie nutritional intervention, which takes about 2 min to inject subcutaneously. The same cat suffering from relocation stress may need only 5 ml of MCT oil for 50-calorie nutritional intervention, which takes about 1 min to inject subcutaneously.

The formulations may be administered shortly before, during, or shortly after the stress to reduce or prevent stress-related weight loss.

Reduction or prevention of weight loss may be detected by measuring the weight of the animal a short time before and some short time after the stress event and/or comparing it to an appropriate control. The short time may be 1 hour, 2 hours, 3 hours, 6 hours, 12 hours, 24 hours, two days, three days, four days, five days, six days, or one week.

The methods prevent weight loss by maintaining the weight of the animal before, during, and after stress substantially unchanged. The weight of the animal shortly before, during, or shortly after the stress remains substantially similar to the pre-stress weight of the animal.

The methods may reduce weight loss in an animal by precluding a loss of between 1% and 50% of the pre-stress weight of the animal. For example, the methods may reduce weight loss by precluding a loss of between 5% and 25% of the pre-stress weight of the animal, such as precluding a loss of about 5%, 6%, 7%, 8%, 9%, 10%, 11%, 12%, 13%, 14%, 15%, 16%, 17%, 18%, 19%, 20%, 21%, 22%, 23%, 24%, of 25% of the pre-stress weight of the animal.

Weight loss may be reduced by between 1%, 5%, 10%, 15% and 50%, 60%, 70%, 80%, 90%, 95% relative to the stress-related control weight. Weight loss can be reduced by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% relative to the stress-related control weight. A reduction in weight loss of about 95%, 96%, 97%, 98%, 99%, or 100% may be prevention of weight loss, wherein the weight of the animal under stress, or just after stress event, is substantially similar to the stress-free control weight.

B. Animals to be Treated

The veterinary community has made a strong clinical impression associating anticipated recovery and weight gain in injured animals. However, a one-size-fits-all application of this theory has had unintended consequences for injured animals. Surgeons frequently avoid the use of front-line opiate analgesics to provide pain management for their animal patients. Numerous studies have reported weight loss in laboratory animals treated with opiate analgesia.

The formulations and methods reduce or prevent weight loss in animals due to stress, including surgery, opiate analgesia, and other stress events. The animals in need include amphibians, reptiles, birds, and mammals having a weight between 0.01 kg (0.02 lb) and 30 kg (66 lb). The animals in need may be agriculture animals, laboratory animals, zoo animals, domestic pets, animals in the wild, and rescue animals.

Specific examples of agriculture animals, such as farm animals, that may benefit from the rapid nutritional intervention method include poultry, such as chickens, hens, roosters, turkeys, ducks, geese, pheasants, quail, and swans, piglets, lamb, kid, goat, rabbits, hares, puppies, dogs, kittens, cats, and foals.

Specific examples of laboratory animals that may benefit from the rapid nutritional intervention method include chickens, hens, roosters, piglets, lamb, kid, goat, rabbits, puppies, dogs, kittens, cats, mice, rats, ferrets, hamsters, gerbil, guinea pig, and non-human primates.

The zoo animals that may benefit from the rapid nutritional intervention method include amphibians, reptiles, birds, and mammals having a weight between 0.01 kg (0.02 lb) and 30 kg (66 lb). These include newborns to larger adult mammals, such as adult mammals weighing more than 30 kg. These include newborns of seals, zebras, tigers, leopards, pandas, and non-human primates.

Specific examples of domestic pets that may benefit from the rapid nutritional intervention method include puppies, dogs, kittens, cats, mice, rats, ferrets, guinea pigs, hamsters, squirrels, hedgehogs, parrots, doves, amphibians including frogs, newts, salamanders, and reptiles including lizards, alligators, chelonians, and snakes.

Animals in the wild may also benefit from the methods. The animals in the wild monitored for habitat use, or migration habits, and identified as in distress may receive the rapid nutritional intervention.

An animal in need may also be a rescued farm animal, laboratory animal, zoo animal, or a domestic pet. The method may be applied to reduce or prevent weight loss in a distressed animal following rescue.

Typically, the methods and formulations are used to reduce or prevent stress-related weight loss. The stress-related weight loss may be due to a drop in appetite, inability to feed, or incapacitation. The stress may be caused by any action, such as animal restrain, surgery, injury, transportation, relocation, birth, separation, exercise, training, handling, and rescue.

### IV. Kits

Kits containing the formulations in dosage forms, in vials, or in dosage form and vials are also provided. The kits may include a reference chart with animal weights, suitable dosages, and associated calories that may be provided by the formulations.

Kits can contain ready-to-use dosage forms, such as sterile syringes pre-loaded with the formulations. The ready-to-use sterile syringes may have a volume of 0.3 milliliters (ml), 0.5 ml, 1 ml, 2 ml, 2.5 ml, 3 ml, 5 ml, 6 ml, 10 ml, 12 ml, 20 ml, 50 ml, 60 ml, 70 ml, or 100 ml. The kits may also contain sterile disposable needles, such as needles ranging in size between 10 gauge (G) and 31 G, or between 16 G and 25 G. Each syringe may provide an individual dose of the formulation, such as a dose between 0.1 ml/kg and 15 ml/kg of the formulation.

Kits can contain septum capped vials holding between 1 and 500 ml of the formulations. The kits may also include sterile disposable needles and sterile syringes, such as sterile needles having a size between 10 G and 31 G and sterile syringes having a volume between 0.3 ml and 100 ml.

The present invention will be further understood by reference to the following non-limiting examples.

### Examples

### Example 1. Fast Supply of 100 Calories of Nutritional Energy to Medium Size Animals

Materials and Methods

Infusion rates and supply of calories per minute were compared between the standard IV infusions of isotonic 5% dextrose solutions (D5W) (provides 4 calories per gram) and MCT oil (provides 9 calories per gram). Table 2 below summarized the infusion conditions and the time needed to achieve 100 calories of nutritional energy.

Results

Compared to standard IV and SC infusions, the value of nutritional support provided by a SC injection of MCT oil is extraordinary. Table 2 illustrates the time and infusion volume needed to supply 100 calories of nutritional support to a dog or a cat treated with typical IV infusions of isotonic 5% dextrose solutions (D5W).

TABLE 2
Infusion rates of D5W v. MCT to achieve 100 calories of nutritional
energy.

| | Volume D5W^{a} to | Time | | Time, MCT^{c} |
|---|---|---|---|---|
| Animal | supply 100 | at 20 mL/ | Time at | injection |
| (wt in lbs) | calories | hour^{b} | 50 mL/hr | 10 mL |
| Cat (15 lbs) | 500 mL | 25 hrs. | n/a | 2 min. |
| Dog (50 lbs) | 500 mL | n/a | 10 hrs | 2 min. |

^{a}Dextrose in isotonic 5% dextrose solutions = 4 calories per gram
^{b}Because the cat is smaller than the dog, smaller veins require slower infusion rates to protect venous access
^{c}MCT = 9 calories per gram

### Example 2. Sterile MCT Oil is Stable at 25° C. and at 40° C.

Materials and Methods

United States Pharmacopeia (USP) grade MCT oil can be bottled in U.S. Food and Drug Administration (FDA)-acceptable serum-capped vials. Although the vial filling process was conducted aseptically and sterility tests revealed no detectable contaminants, the oil was treated with 1% benzyl alcohol preservative as required by FDA's standards for multi-use vials. The vials were then sterilized by gamma radiation. This process is compatible with FDA's Good Manufacturing Practices (GMP).

Tested Parameters:

BA, benzyl alcohol, is used as a preservative, and its content is measured by HPLC (high pressure liquid chromatography).

Viscosity is measured using a rotational viscometer, which measures the torque required to turn an object in a fluid as a function of that fluid's viscosity.

Conform: This term is used in establishing manufacturing and laboratory standards. The term establishes a contractual obligation with the user and an agency that monitors the manufacturer/laboratory. To illustrate, the manufacturer buys a standard grade ingredient to make product. The ingredient is furnished with certificates that it has met or exceeded standards for purity, content, and stability. The user does not have a laboratory at his/her plant to check the laboratory values. However, the user can/should establish in concert with the supplier “conformity” values. For example, regardless of a certification that the product is 100 percent pure, the powder should be white, it should be free of lumps; it should be free of debris; and so on. If it does not meet these agreed upon expectations, the product does not conform and it is rejected. In addition, a “non-conformity” claim should be followed by established trouble shooting protocols and corrective actions.

Syringeability: This is a term to assure that a liquid, gel, or semi-solid product can be injected via a syringe and needle. The term is frequently combined with the volume of the syringe and gauge of the needle. For example, some oils can be delivered easily through a large gauge needle (14-20 ga.), but practically cannot be delivered through a smaller 26-28 ga. needle.

Recently, manufactures of opiate medicines and other drugs of abuse have claimed their oral tablets cannot be crushed and delivered via a syringe (Non-syringeable) product, a common practice in illegal drug use and diversion.

Resuspendability: This term is assigned to a product with a claim to be a uniform suspension. During manufacturing, solids are uniformly blended with liquids to generate a uniform suspension. If the solids settle out of the suspension during storage, the manufacturer claims that uniformity can be reestablished by gentle to vigorous shaking, by hand or machine, and for seconds to minutes of more. If the uniform suspension cannot be established by the resuspension instructions, the product does not conform.

For example, ANIMALGESICS® (Baltimore Animal Medicine, Inc., MD) for mice and rats is a suspension of cholesterol, triglyceride, and buprenorphine in medium chain triglyceride oil. The user is instructed to shake the vial for 30 seconds to insure the uniform suspension is reestablished. Note, the terms “uniformity” and “homogeneity” can convey different standards depending on the product.

Sterility: Product is sampled and tested by a laboratory to measure bacterial and fungal contamination. Generally, a mL of product is spread on or inoculated into gels that are primed to grow fungus or bacteria. The inoculum is heated to body temperature for one or more days to determine whether it has contaminating microorganisms that are detectable by growth in the laboratory inoculum. Sterility, which is a statistical concept, is reported as no growth, or one or more colonies grown by a certain period.

Results

Data from the stability studies of vials stored at room temperature (25°) and accelerated stability test conditions, storage at 40°, are shown in Tables 3 and 4.

These studies demonstrate that the formulations and vials can be made by the GMP standards required by the FDA for sale as a parenteral nutritional supplement for animals and humans.

TABLE 3
Stability of Radiation Sterilized MCT in Septum-Capped Drug
Vials with 1% Benzyl Alcohol Preservative Stored at 25° C.

| | | | Pull Date: | |
|---|---|---|---|---|
| | | | 20-Sep- | 20-Dec- |
| | | | 16 | 16 |
| | | T/H^{a} | | |
| | | n/a | 25/60 | 25/60 |
| Attribute | Specification | t = 0^{b} | 1M^{c} | 3M |
| Description | Conforms | Conforms | | Conforms |
| ID | Conforms | Conforms | | Conforms |
| BA Assay | 0.83-1.01% | 0.92 | | 0.90 |
| | v/v | | | |
| Viscosity | 48-130 cP | 53 | | 49 |
| Resuspend | | 26 | | 31 |
| ability | | | | |
| Syringe | Conforms | Conforms | | n/a |
| ability | | | | |
| Sterility | Conforms | n/a | | n/a |

^{a}T/H, temperature (° C.)/humidity (%)
^{b}t = 0, at time 0
^{c}1M, at 1 month from t = 0

TABLE 4
Stability of Radiation Sterilized MCT in Septum-Capped Drug
Vials with 1% Benzyl Alcohol Preservative Stored at 40° C.

| | | | Pull Date: | |
|---|---|---|---|---|
| | | | 20-Sep- | 20-Dec- |
| | | | 16 | 16 |
| | | T/H | | |
| | | n/a | 40/75 | 40/75 |
| Attribute | Specification | t = 0 | 1M | 3M |
| Description | Conforms | Conforms | Conforms | Conforms |
| ID | Conforms | Conforms | Conforms | Conforms |
| BA Assay | 0.83-1.01% | 0.92 | 0.91 | 0.90 |
| | v/v | | | |
| Viscosity | 48-130 cP | 53 | 68 | 65 |
| Resuspend | | 26 | n/a | 33 |
| ability | | | | |
| Syringe | Conforms | Conforms | n/a | n/a |
| ability | | | | |
| Sterility | Conforms | n/a | n/a | n/a |

### Example 3. MCT Oil Maintains Animal's Body Weight During Post-Surgery Recovery with an Analgesic

Materials and Methods

Body weights were measured in 4 groups of surgically-treated female rats. The groups included the control group—opiate free drug solution, and test groups with increasing doses (1.3-6.5 mg/kg buprenorphine) of the appetite-suppressant opiate drug, buprenorphine. The weights were obtained by a technician blinded to the treatment group. Surgery plus anesthesia is known to suppress appetite and weight gain. Opiate analgesia further suppresses appetite by opiate-binding and inhibiting cell activity of the nerve cells lining the gut. This inhibition blocks normal gut movement (peristalsis) the action which is thought to suppress appetite.

Results

The results are presented in Table 5.

TABLE 5
Post-Surgery Body Weight Gains (BWG) for Female Rats Given
Increasing Doses of Opiate Buprenorphine and Increasing Volumes of MCT.
Data are presented as Mean ± Standard Deviation (SD).

| Post-Surgery Dose | BWG | BWG | BWG | | BWG |
|---|---|---|---|---|---|
| Group | Day | Day | Day | | Day |
| Buprenorphine | 0-4 | 4-6 | 6-8 | BWG Day 8-12 | 0-12 |
| Control | 2.1 ± 2.1 | 2.2 ± 2.4 | 1.2 ± 3.0 | 6.4 ± 1.2 | 9.6 ± 2.0 |
| 0 mg/kg | | | | | |
| (1.0 ml MCT) | | | | | |
| 1.3 mg/kg | −1.9 ± 0.3 | 2.0 ± 0.9 | −3.4 ± 3.5 | 6.7 ± 1.4 | 3.4 ± 5.1 |
| (0.2 mL MCT) | | | | | |
| 3.9 mg/kg | −3.2 ± 1.5 | 3.8 ± 2.6 | −2.7 ± 1.2 | 6.7 ± 5.4 | 4.6 ± 6.3 |
| (0.6 mL MCT) | | | | | |
| 6.5 mg/kg | −1.5 ± 3.5 | 3.4 ± 1.9 | −1.8 ± 2.7 | 7.0 ± 5.8 | 7.0 ± 6.2 |
| (1.0 mL MCT) | | | | | |

As shown in the top row of data, there are positive weight changes of the control group given 1.0 mL of opiate-free MCT solution (the placebo control group). The experimental rats fail to lose weight due to the ca. 100 calorie subcutaneous (SC) infusion of 1.0 mL of MCT. The nutritional value of the MCT oil is shown in the second row of animals given the gut inhibiting opiate at a dose of 1.3 mg/kg. These rats gain approximately 3.4 grams of weight by day-12 post surgery, anesthesia, and opiate analgesia. The rats given 5 times the dose of opiate, 6.5 mg/kg, gain a remarkable 7.0 grams by day-12 post surgery. The increasing SC administration of MCT, from 0.2 to 1.0 mL, completely compensate for the appetite suppression caused by the higher doses of opiate.

This example demonstrates the unexpected result that the formulation can be used to provide a bolus of calories independent of drug, and that the formulation can be used to compensate for drug induced loss of appetite.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A method for reducing or preventing stress-related or drug induced weight loss in an animal, the method comprising subcutaneously administering to an animal in need thereof a formulation comprising sterile medium-chain triglyceride oil as the active agent.

2. The method of claim 1, wherein administering occurs shortly before, during, or shortly after the stress.

3. The method of claim 1, wherein the formulation is administered over a period of time ranging from two seconds to two minutes.

4. The method of claim 1, wherein the animal is a small to medium size animal ranging in weight between 0.01 kg (0.02 lb) and 30 kg (66 lb).

5. The method of claim 1, wherein the animal is selected from the group consisting of a laboratory animal, veterinary animal, zoo animal, and a pet.

6. The method of claim 1, wherein the stress is due to any action selected from the group consisting of animal restrain, surgery, injury, transportation, relocation, birth, separation, exercise, training, handling, and rescue.

7. The method of claim 1, wherein the formulation is administered to the dorsal mid scapular scruff.

8. The method of claim 1, wherein the weight of the animal shortly before, during, or shortly after the stress remains substantially similar.

9. The method of claim 1, wherein preventing weight loss is preventing a loss of between 1% and 50% of pre-stress weight.

10. A formulation for use in the method of claim 1.

11. The formulation of claim 10, wherein the sterile medium-chain triglyceride oil comprises triglycerides wherein at least one fatty acid chain has a length between six (C₆) and twelve (C₁₂) carbons.

12. The formulation of claim 10, wherein the medium-chain triglyceride oil is a first active agent and the formulation further comprises a second active agent.

13. The formulation of claim 10, wherein the second active agent is selected from the group consisting of vitamins, minerals, antibiotics, and anti-parasiticals.

14. The formulation of claim 12, comprising at least 50% by weight medium-chain triglyceride oil.

15. The formulation of claim 10, wherein formulation comprises at least 50% by weight medium-chain triglyceride oil and does not include a second active agent.

16. The formulation of claim 10 wherein the formulation comprising an analgesic that suppresses appetite, which is not buprenorphine.

17. The formulation of claim 10 further comprising essential nutrients including poly unsaturated fatty acids and/or natural or synthetic d/1 amino acids.

18. A kit comprising ready-to-use sterile syringes pre-loaded with a dosage of the formulation of claim 10.

19. The kit of claim 18, wherein the ready-to-use sterile syringes have a volume selected from the group consisting of 0.3 milliliters (ml), 0.5 ml, 1 ml, 2 ml, 2.5 ml, 3 ml, 5 ml, 6 ml, 10 ml, 12 ml, 20 ml, 50 ml, 60 ml, 70 ml, and 100 ml.

20. The kit of claim 18, wherein the kit comprises sterile needles ranging in size between 10 gauge (G) and 31 G, or between 16 G and 25 G.

21. The kit of claim 18, wherein the dosage delivers between 0.1 ml/kg and 15 ml/kg of MCT oil.

22. A kit comprising one or more septum capped vials comprising between 5 and 500 ml of the formulation of claim 10.

23. The kit of claim 22, further comprising one or more sterile needles and sterile syringes.

24. The kit of claim 23, wherein the needles have a size between 10 G and 31 G and the syringes have a volume between 0.3 ml and 30 ml.
